(19) United States
(12) Patent Application Publication
(10) Pub. No.: US 2005/0246864 A1
(43) Pub. Date: Nov. 10, 2005

(54) Hinge device

(75) Inventor: Masaru Arakawa, Suita-shi (JP)

Correspondence Address: HAUPTMAN KANESAKA BERNER PATENT AGENTS, SUITE 300, 1700 DIAGONAL RD, ALEXANDRIA, VA, 22314-2848, US

(73) Assignee: NIFCO INC., Yokohama-shi (JP)

(21) Appl. No.: 11/102,768

(22) Filed: Apr. 11, 2005

(30) Foreign Application Priority Data
Apr. 15, 2004 (JP) 2004-120070

(51) Int. Cl. A61F 2/18

(52) U.S. Cl. 16/277

(57) ABSTRACT

A hinge device rotationally connects a first member and a second member, and is capable of stopping one of the first member and the second member at an arbitrary angle. The hinge device includes a coil spring having a wound part and a coupling part provided on one end of the wound part, and a cylindrical spring member with a C-shaped section. The cylindrical spring member has a slit extending in a longitudinal direction thereof, and an outer diameter larger than an inner diameter of the wound part. The cylindrical spring member is retained in the wound part in a contracted state. The coupling part of the coil spring is connected to one of the first member and the second member, and an engaging part protruding from an end of the cylindrical spring member is connected to the other of the first member and the second member.

### BACKGROUND OF THE INVENTION AND RELATED ART STATEMENT

The present invention relates to a hinge device used in a device such as a notebook PC and an electronic dictionary that can be folded. In particular, the present invention relates to a free stopping type hinge device that stops a member at an arbitrary angle in a course of rotation and maintains a posture at the angle.

A free stopping type hinge device is applied to a device such as a notebook PC. The hinge device is disposed between a rotating body including a display and a main body, so that the rotating body can rotate relative to the main body and be held at an arbitrary angle. In this structure, a wound part of a coil spring is tightly wound on a fixed shaft on the main body for applying sliding resistance between the coil spring and the fixed shaft. Both ends of the coil spring are fixed to a rotating shaft provided on the rotating body. Accordingly, it is possible to hold the rotating body at an angle after adjustment through the sliding resistance between the coil spring and the fixed shaft (see Patent Document 1).

Patent Document 1: Japanese Patent Publication (Kokai) No. 2000-45614

In the conventional structure, the sliding resistance between the coil spring and the fixed shaft is obtained with the wound part of the coil spring closely wound on the fixed shaft. It is possible that a constituent member such as the wound part wears through continuous use, thereby changing the sliding, resistance. When the resistance gradually becomes small, it is possible that the rotating body moves under own weight due to vibration and the like.

In view of the problems described above, an object of the present invention is to provide a hinge device with a simple structure, in which it is possible to stop a member at an arbitrary angle with constant sliding resistance even when a member wears.

Further objects and advantages of the invention will be apparent from the following description of the invention.

### SUMMARY OF THE INVENTION

In order to achieve the objects mentioned above, according to a first aspect of the present invention, a hinge device connects a first member and a second member to be rotatable and is capable of stopping one of the first member and the second member an arbitrary angle. The hinge device comprises a coil spring having a wound part and a coupling part provided on one end of the wound part; and a cylindrical spring member with a C-shaped section. The cylindrical spring member has a slit extending in a longitudinal direction of the cylindrical spring member, and an outer diameter larger than an inner diameter of the wound part. The cylindrical spring member is retained in the wound part in a contracted state. The coupling part of the coil spring is connected to one of the first member and the second member, and an engaging part protruding from an end of the cylindrical spring member is connected to the other of the first member and the second member.

In the hinge device, sliding resistance is generated between both members in a state in which the cylindrical spring member is inserted into the wound part of the coil spring in a contracted state. Even when the members such as the wound part wear, the sliding resistance is corrected by a springing-back force via the slit of the cylindrical spring member, and is maintained at a value same as an initial setting. When the wound part of the coil spring is rotated in a direction opposite to a winding direction of the wound part, the sliding resistance becomes weaker as the wound part is unwound. Conversely, when the wound part is rotated in the same direction as the winding direction of the wound part, the wound part is wound up and the sliding resistance is increased. In this case, the sliding resistance can be stabilized and adjusted by placing an intermediary substance such as oil between the wound part of the coil spring and the cylindrical spring member. The present invention may be modified as follows.

According to the present invention, one of the first member and second member has a bracket part protruding from a corresponding end side; a storage cavity provided in the bracket part; and a shaft part protruding from an outer end surface of the bracket part. The storage cavity receives the coil spring and the cylindrical spring member, and is capable of engaging the coupling part. The other of the first member and second member is connected to the shaft part to be rotatable freely, and is connected to the coupling part of the cylindrical coil spring not to be rotatable.

According to the present invention, one of the first member and second member has a bracket part protruding from a corresponding end side; an engaging hole provided in an inner end surface of the bracket part for inserting the coupling part of the coil spring; a guiding shaft part inserted into the inner diameter of the cylindrical spring member; and a supporting shaft part protruding on an outer end surface of the bracket part. The other of the first member and second member is connected to the supporting shaft part to be rotatable freely, and is connected to the coupling part of the cylindrical coil spring not to be rotatable.

According to the present invention, one of the first member and second member has a bracket part protruding from a corresponding end side; a storage cavity provided in the bracket part; and a shaft hole formed in an outer end surface of the bracket part. The storage cavity receives the coil spring and the cylindrical spring member, and is capable of engaging the coupling part. The other of the first member and second member is connected to a shaft part to be inserted into an inner diameter of the cylindrical spring member through the shaft hole to be rotatable freely, and is connected to the coupling part of the cylindrical coil spring not to be rotatable.

The present invention provides the following advantages. The cylindrical spring member having elasticity by the slit is pressed in the wound part. Accordingly, when the wound part and the cylindrical spring member wear, the sliding resistance is adjusted by the springing-back force of the cylindrical spring member, thereby reducing a variation due to the wear and maintaining a stable operation. Further, it is possible to modify the structure of the hinge device according to the structures of the first member and second member, thereby improving design and reliability.

### BRIEF DESCRIPTION OF THE DRAWINGS

FIGS. 1(a) and 1(b) are perspective views showing a device according to a first embodiment of the present invention;

FIGS. 2(a) and 2(b) are views showing essential components of the device shown in FIG. 1(a);

FIGS. 3(a) and 3(b) are exploded views of the essential components of the device shown in FIG. 1(a);

FIG. 4(a) is a sectional view of a device and FIG. 4(a) is an exploded view thereof according to a second embodiment of the present invention; and

FIG. 5(a) is a sectional view of a device and FIG. 5(b) is an exploded view thereof according to a third embodiment of the present invention.

### DETAILED DESCRIPTION OF PREFERRED EMBODIMENTS

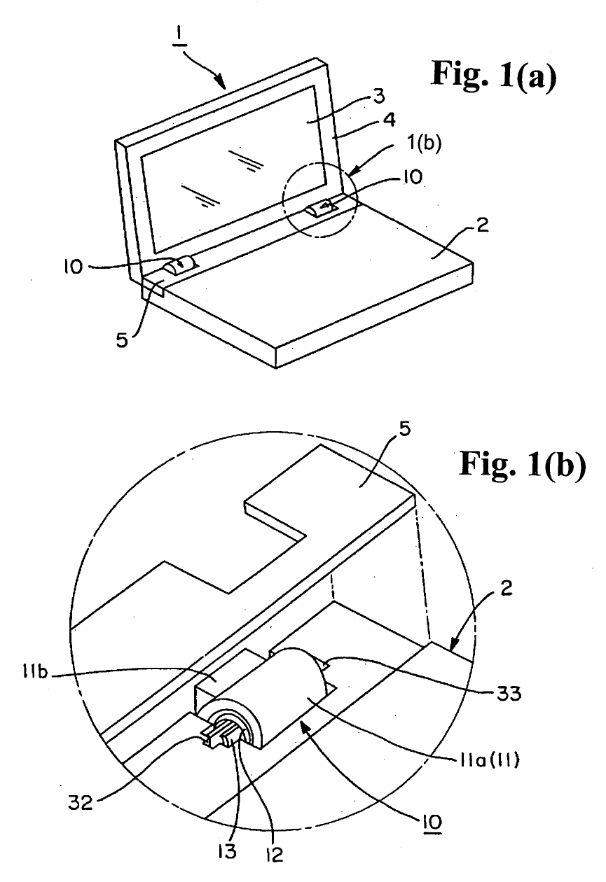

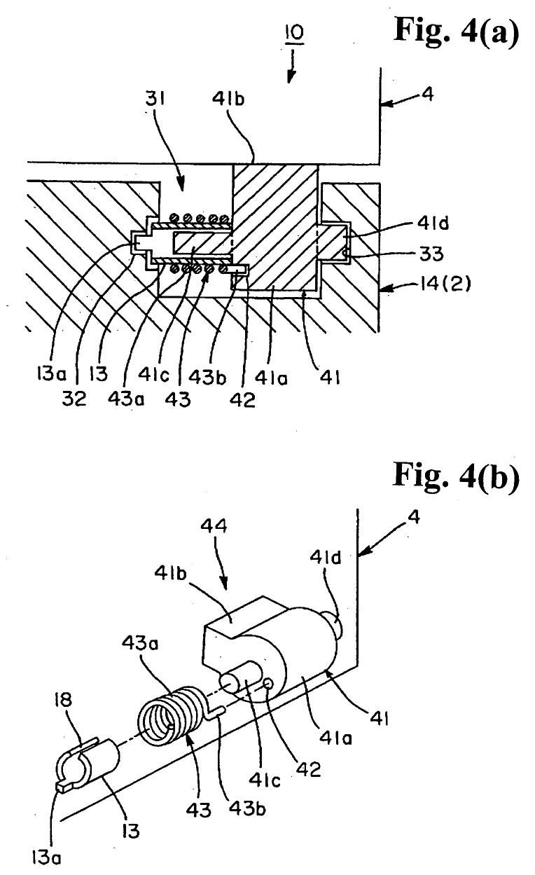

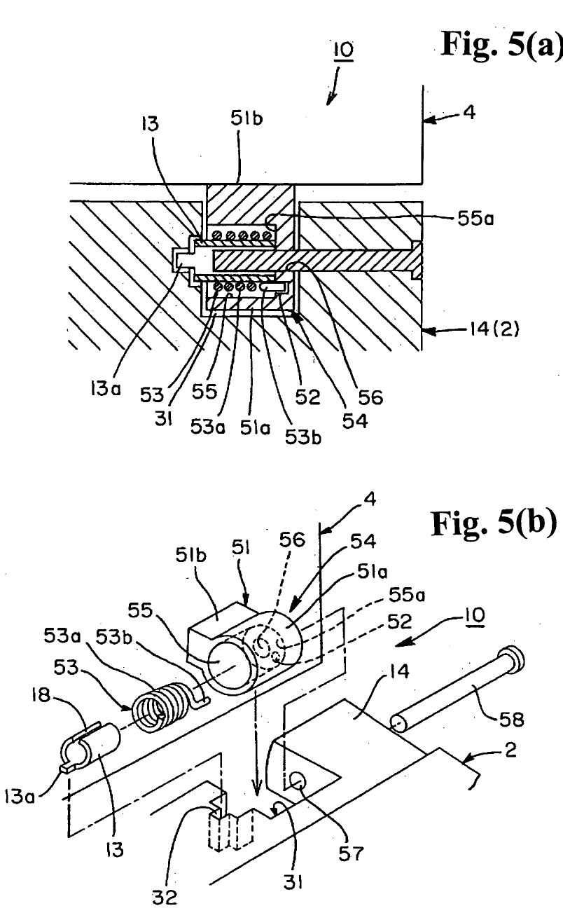

Hereunder embodiments of the present invention will be explained with reference to the accompanying drawings. FIGS. 1(a)-1(b) to 3(a)-3(b) show a first embodiment, FIGS. 4(a) and 4(b) show a second embodiment, and FIGS. 5(a) and 5(b) show a third embodiment. In the explanation, after describing an outline of a device of the invention, the first embodiment, second embodiment, and third embodiment will be described in detail successively. In the drawings, the same symbols are assigned to members and parts that are the same in operation.

(Outline)

As shown in a notebook PC 1 in FIG. 1, a free stopping type hinge device 10 of the present invention connects a rotating body 4 to be capable of rotation on a device main body 2, and holds the rotating body 4 at an arbitrary angle after rotation. A purpose of improvement is to make it possible to maintain almost the same amount of sliding resistance for free stopping as at the initial time, being made unlikely to be affected even when the constituent members wear through long-term use.

An essential component structure comprises a coil spring 12 having a wound part 12a and a coupling part 12b provided on one end of the wound part, and a cylindrical spring member 13 with a roughly C-shaped section having a slit 18 in a longitudinal direction of the cylinder, and is made having an outer diameter larger than the inner diameter of the wound part 12a, and is pressed in a contracted state into the wound part 12a. Also, the coupling part 12b of the coil spring 12 is connected to one of the main body 2 and rotating body 4, and a coupling piece part 13a protruding from a corresponding end of the cylindrical spring member 13 is connected to the other.

Hereunder, structures of the first embodiment to the third embodiment will be described. In each embodiment, in the notebook PC 1 in FIG. 1, the rotating body 4 is connected on the main body 2 by left and right: free stopping type hinge devices 10, such that the rotating body 0.4 is set to a prescribed angle during use, and the rotating body 4 is folded on top of the main body 2 when not in use.

### First Embodiment

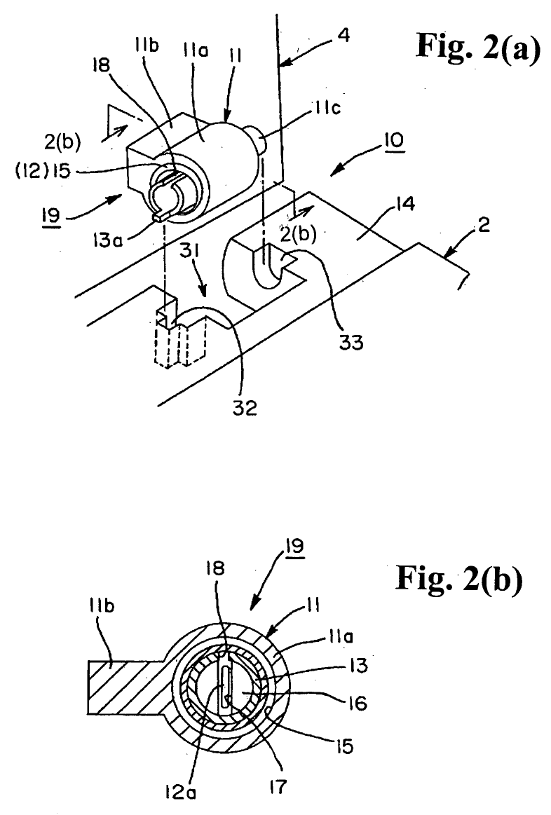

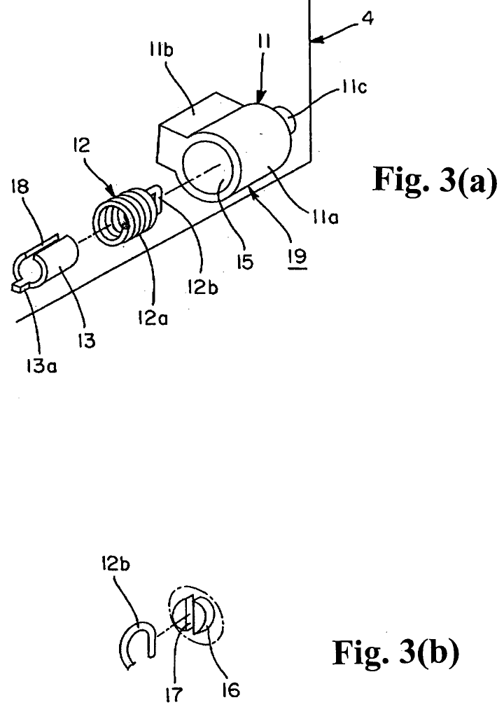

FIG. 1(a) shows the notebook PC to which the present invention is applied, and FIG. 1(b) is an enlarged view of a part 1(b). FIG. 2(a) is a view showing a relationship among the hinge device, the main body, and the rotating body, and FIG. 2(b) is a sectional view taken along line 2(b)-2(b) in FIG. 2(a). FIG. 3(a) is an exploded view of an essential component, and FIG. 3(b) is a view showing a structure in which one end of a coil spring is connected to a bracket part.

The hinge device 10 is constituted by a coil spring 12 having a wound part 12a; a cylindrical spring member 13 pressed in a contracted state into the wound part 12a; a bracket part 11 protruding from the rotating body 4 and received in the coil spring 12 together with the cylindrical spring member 13; and a fixed shaft bearing part 14 provided on the main body 2 and connected with the bracket part 11.

The bracket part 11 has a main part 11a integrated with an attachment part 11b provided on a front lower side of the rotating body 4 to form a storage cavity 15 having a cylindrical shape with a bottom. On an outer end surface of the main part 11a a supporting shaft part 11c is provided coaxially with the storage cavity 15. In the inner end surface 16 of a recessed wall inside the storage cavity 15, a slot-like engaging part 17 is formed for engaging a coupling part 12b on one end side of the coil spring 12 as shown in FIG. 3(b).

The coil spring 12 has a wound part 12a wound in a ring shape. An outer diameter of the wound part 12a is smaller than an inner diameter of the storage cavity 15, and the coil spring 12 is entirely received inside the storage cavity 15. A coupling part 12b bent in the diameter direction is provided on one end side of the wound part 12a. The coupling part 12b is fixed to be incapable of rotation in a state being coupled in the slot of the engaging part 17 when the coil spring 12 is placed inside the storage cavity 15.

The cylindrical spring member 13 has a roughly C-shaped section with a gap (slit 18) between both ends. The cylindrical spring member 13 is made of a resin by molding or a steel material by bending an elastic plate into a cylindrical shape. A length of the cylindrical spring member 13 is the same as or somewhat larger than a length of the wound part 12a in the axis direction. The outer diameter (outer diameter when not pressed into the wound part 12a) of the cylindrical spring member 13 is larger than the inner diameter of the wound part 12a. A coupling piece part 13a elongated outwardly along the cylinder is formed on one end side of the cylindrical spring member 13.

When the cylindrical spring member 13 is assembled with the coil spring 12, for example, the coupling part 12b on one end of the coil spring 12 is fixed and the other end side is rotated by a tool, or the like, in the direction of unwinding of the wound part 12a, that is, up to a position where the inner diameter of the wound part 12a becomes greater than the outer diameter of the cylindrical spring member 13. From this state, the cylindrical spring member 13 is inserted into the wound part 12a from the direction of placement in FIG. 3(a). When the other end is released, the wound part 12a is pressed against the cylindrical spring member 13, and the cylindrical spring member 13 is attached to the coil spring 12. In the attached state, the cylindrical spring member 13 is projecting the coupling piece part 13a from the corresponding end of the wound part 12a. The sliding resistance is generated between the wound part 12a and the cylindrical spring member 13.

The coil spring 12 assembled with the cylindrical spring member 13 is inserted into the storage cavity 15 from the side of the coupling part 12b, and the coupling part 12b engages the engaging part 17, so that the coil spring 12 is assembled to the bracket part 11 (rotating body 4). When the bracket part 11 is a housing to be attached to the rotating body 4, the free stopping type hinge device 10 is handled as a unit 19 formed of the bracket part 11, coil spring 12, and cylindrical spring member 13.

A cut-out part 31 is provided in the fixed shaft bearing part 14 for receiving the bracket part 11. A turn-stopping cavity 32 for receiving the coupling piece part 13a of the cylindrical spring member 13 protruding from the storage recess 15 to be incapable of rotation, and a shaft bearing cavity 33 for receiving the shaft part 11c of the bracket part 11 to rotate freely are provided in opposite sides of the cut-out part 31.

In the shaft bearing part 14, as shown in FIG. 2(a), the coupling piece part 13a of the cylindrical spring member 13 is coupled into the cavity 32, and the shaft part 11c is coupled into the shaft bearing cavity 33, in a state that the main part 11a of the bracket part 11 corresponds to the cut-out part 31. In this state, the cylindrical spring member 13 becomes incapable of rotation on the fixed shaft bearing part 14, and the bracket part 11 together with the coil spring 12 becomes capable of rotation via the shaft part 11c. The winding direction of the wound part 12a is the same as the closing direction of the rotating body 4. A cover 5 is attached to the main body 2 in correspondence with the fixed shaft bearing part 14 as shown in FIG. 1(b). Accordingly, the bracket part 11 is installed in the cut-out part 31, while the cavity 32 and shaft bearing cavity 33 are covered with the cover 5, thereby making the shaft part 11c and coupling piece part 13a invisible.

(Operation)

An operation of the rotating body 4 with the free stopping type hinge device 10 will be explained next. First, the rotating body 4 is folded on top of the main body 2 by rotating in the winding direction of the wound part 12a with the shaft part 11c and cylindrical spring member 13 as fulcrum. In this process, the wound part 12a is pressed against the cylindrical spring member 13, so that the sliding resistance is generated between that wound part 12a and the cylindrical spring member 13. In order to open the rotating body 4 in-use state from the closed state in which the rotating body 4 is folded onto the main body 2, the front end of the rotating body 4 is lifted by a hand. The rotating body 4 rotates upwardly with the shaft part 11c and cylindrical spring member 13 as fulcrum. In this process, the wound part 12a is rotated in the direction of unwinding the winding part via the coupling part 12b, so that the sliding resistance between the wound part 12a and the cylindrical spring member 13 is reduced. The rotating body 4 is held in the tilted state at a desired angle by the sliding resistance even when the hand is removed from that rotating body 4, i.e., a free stopping state.

The cylindrical spring member 13 is pressed in a compressed or contracted state into the wound part 12a, and the springing-back force is always provided between the cylindrical spring member 13 and the wound part 12a. Accordingly, even if the wound part 12a and cylindrical spring member 13 wear, the sliding resistance is adjusted by the springing-back force via the slit 18 of the cylindrical spring member 13, thereby maintaining the sliding resistance at the initial time and stabilizing the sliding resistance for a long time.

### Second Embodiment

FIGS. 4(a) and 4(b) show the second embodiment, wherein FIG. 4(a) is a sectional view of an essential component of a device, and FIG. 4(b) is an exploded view of the essential component of the device. The hinge device 10 has a coil spring 43 having a wound part 43a; a cylindrical spring member 13 pressed in a contracted state into the wound part 43a; a bracket part 41 protruding from the rotating body 4; and a fixed shaft bearing part 14 provided in the main body 2 and connected with the bracket part 41.

The bracket part 41 has a main part 41a integrated with an attachment part 41b fixed to a front lower side of the rotating body 4, and integrally has an inside shaft part 41c protruding from a center of one end surface and an outside shaft part 41d protruding from a center of the other end surface. In one end surface of the bracket part 41, a spring engaging hole 42 is provided adjacent to the inside shaft part 41c.

The coil spring 43 has a wound part 43a wound as a ring. On one end side of the wound part 43a, a coupling part 43b is placed extending in the axial direction. The coupling part 43b is inserted into the engaging hole 42 in a state in which the coil spring 43 has the shaft part 41c inserted into the wound part 43a. In the coil spring 43, the cylindrical spring member 13 is pressed in an inner diameter of the wound part 43a similar to the first embodiment.

When the coil spring 43 assembled with the cylindrical spring member 13 is placed in the corresponding part of the main part 41a such that the inside shaft part-41c is inserted through the cylindrical spring member 13 and the coupling part 43b is inserted into the engaging hole 42, the coil spring 43 and the cylindrical spring member 13 are integrated on the bracket part 41. When the bracket part 41 is a housing to be attached to the rotating body 4, the free stopping type hinge device 10 is handled as a unit product 44 formed of the bracket part 41, coil spring 43, and cylindrical spring member 13.

A cut-out part 31 is provided in the fixed shaft bearing part 14 for receiving the bracket part 41 and the coil spring 43. A turn-stopping cavity 32 for receiving the coupling piece part 13a of the cylindrical spring member 13 to be incapable of rotation and a shaft bearing cavity 33′ for receiving the shaft part 41d of the bracket part 41 to rotate freely are provided in opposite sides of the cut-out part 31. The cavity 32 and the cavity 33 are closed by a cover (not shown) similar to the first embodiment.

In the fixed shaft bearing part 14, the coupling piece part 13a of the cylindrical spring member 13 is coupled into the cavity 32, and the shaft part 41d is coupled into the shaft bearing cavity 33 in a state that the main part 41a of the bracket part 41 corresponds to the cut-out part 31. In this state, the cylindrical spring member 13 becomes incapable of rotation on the fixed shaft bearing part 14, and the bracket part 41 together with the coil spring 43 becomes capable of rotation via the shaft part 41d. The winding direction of the wound part 43a is the same as the closing direction of the rotating body 4. A cover is attached to the main body 2 in correspondence with the fixed shaft bearing part 14 as in the first embodiment. Accordingly, the bracket part 41 is installed in the cut-out part 31 while the cavity 32 and shaft bearing cavity 33 is closed with the cover, thereby making the coupling piece part 13a and the shaft part 41d invisible.

(Operation).

An operation of the rotating body 4 with the free stopping type hinge device 10 is almost the same as that of the first embodiment. That is, the rotating body 4 is folded on top of the main body 2 by rotating in the winding direction of the wound part 43a with the shaft part 41d and cylindrical spring member 13 as fulcrum. In this process, the wound part 43a is wound up in a state being pressed against the cylindrical spring member 13, and the sliding resistance generated between that wound part 43a and the cylindrical spring member 13 increases. In order to open the rotating body 4 in-use state from the closed state in which the rotating body 4 is folded onto the main body 2, the front end of the rotating body 4 is lifted by a hand. Accordingly, the rotating body 4 rotates upwardly with the shaft part 41d and cylindrical spring member 13 as fulcrum. In this process, the wound part 43a is rotated in the direction of unwinding the winding via the coupling part 43b, and the sliding resistance between the wound part 43a and the cylindrical spring member 13 is reduced. The rotating body 4 is held in the tilted state at the desired angle by the sliding resistance even when the hand is removed from that rotating body 4.

The cylindrical spring member 13 is pressed in a compressed or contracted state into the wound part 43a, and the springing-back force is always provided between the cylindrical spring member 13 and the wound part 43a. Accordingly, even if the wound part 43a and cylindrical spring member 13 wear, the sliding resistance is adjusted by the springing-back force via the slit 18 of the cylindrical spring member 13, thereby maintaining the sliding resistance at the initial time and stabilizing the sliding resistance for a long time.

### Third Embodiment

FIGS. 5(a) and 5(b) show the third embodiment, wherein FIG. 5(a) is a sectional view of an essential component of a device, and FIG. 5(b) is an exploded perspective view of the essential component of the device. The hinge device 10 in FIG. 5 has a coil spring 53 having a wound part 53a; a cylindrical spring 30 member 13 pressed in a contracted state into the wound part 53a; a bracket part 51 protruding from the rotating body 4; and a fixed shaft bearing part 14 provided in the main body 2 and connected with the bracket part 51.

The bracket part 51 has a main part 51a integrated with an attachment part 51b provided on the front lower side of the rotating body 4 to form a storage cavity 55 having a cylindrical shape with a bottom. In the recessed wall part 55a of the storage cavity 55, a through-hole 56 is provided at about the center, and a spring engaging hole 52 is provided adjacent to the through-hole 56.

The coil spring 53 has a wound part 53a wound as a ring, and a coupling part 53b extends from one end side of the wound part 53a in the axial direction. In the coil spring 53, the cylindrical spring member 13 is pressed into the inner diameter of the wound part 53a just as in the first embodiment.

When the coil spring 53 assembled with the cylindrical spring member 13 is inserted into the storage cavity 55 from the side of the coupling part 53b, and the coupling part 53b is checked in the engaging hole 52, the coil spring 53 is assembled on the bracket part 51 (rotating body 4). When the bracket part 51 is a housing to be attached to the rotating body 4, the free stopping type hinge device 10 is handled as a unit product 54 formed of the bracket part 51, coil spring 53, and cylindrical spring member 13.

The cut-out part 31 is provided in the fixed shaft bearing part 14 for receiving the bracket part 51. A turn-stopping cavity 32 for receiving the coupling piece part 13a of the cylindrical spring member 13 to be incapable of rotation and a through-hole 57 for inserting an exclusive shaft member 58 are provided on opposite sides of the cut-out part 31. The shaft member 58 is inserted at the front end side into the through-hole 56 of the storage cavity 55 and the inner diameter of the cylindrical spring member 13 in a state having been inserted through the through-hole 57. The cavity 32 is closed by a cover (not shown) as in the first embodiment.

In the fixed shaft bearing part 14, the coupling piece part 13a of the cylindrical spring member 13 is coupled into the cavity 32 in a state that the main part 51a of the bracket part 51 corresponds to the cut-out part 31. The shaft member 58 is inserted into the through-hole 56 from the through-hole 57, and into the inner diameter of the cylindrical spring member 13. In this state, the cylindrical spring member 13 becomes incapable of rotation on the fixed shaft bearing part 14, and the bracket part 51 becomes capable of rotation via the shaft member 58. The winding direction of the wound part 53a is the same as the closing direction of the rotating body 4. A cover is attached to the main body 2 in correspondence with the fixed shaft bearing part 14 as in the first embodiment. Accordingly, the bracket part 51 is installed in the cut-out part 31 via the cover and the shaft member 58.

(Operation)

An operation of the rotating body 4 with the free stopping type hinge device 10 is almost the same as that in the first embodiment. That is, the rotating body 4 is folded on top of the main body 2 by rotating in the winding direction of the wound part 53a with the shaft member 58 and cylindrical spring member 13 as fulcrum. In this process, the wound part 53a is wound up in a state being pressed against the cylindrical spring member 13, so that the sliding resistance generated between that wound part 53a and the cylindrical spring member 13 increases. In order to open the rotating body 4 in-use state from the closed state in which the rotating body 4 is folded onto the main body 2, the front end of the rotating body 4 is lifted by a hand. Accordingly, the rotating body 4 rotates upwardly with the shaft member 58 and cylindrical spring member 13 as fulcrum. In this process, the wound part 53a is rotated in the direction of unwinding the winding via the coupling part 53b, so that the sliding resistance between the wound part 53a and the cylindrical spring member 13 is reduced. The rotating body 4 is held in the tilted state at the desired angle by the sliding resistance even when the hand is removed from that rotating body 4.

The cylindrical spring member 13 is pressed in a compressed or contracted state into the wound part 53a, and the springing-back force is always provided between the cylindrical spring member 13 and the wound part 53a. Accordingly, even if the wound part 53a and cylindrical spring member 13 wear, the sliding resistance is adjusted by the springing-back force via the slit 18 of the cylindrical spring member 13, thereby maintaining the sliding resistance at the initial time and stabilizing the sliding resistance for a long time.

In the hinge device 10, the sliding resistance is reduced when the rotating body 4 rotates to the in-use state, that is, from the closed to the open position. It may be arranged such that the sliding resistance may increase when the rotating body 4 rotates from the closed to the open position. The application is not limited to the notebook PC 1, and the hinge device can be used in a folding-type electronic dictionary, a portable telephone, and other folded structures.

The disclosure of Japanese Patent Application No. 2004-120070, filed on Apr. 15, 2004, is incorporated in the application.

While the invention has been explained with reference to the specific embodiments of the invention, the explanation is illustrative and the invention is limited only by the appended claims.

1. A hinge device for rotationally connecting a first member and a second member, comprising:
a coil spring having a wound part, and a coupling part provided on one end of the wound part, said coupling part being adapted to be attached to one of the first member and the second member, and
a cylindrical spring member having a slit extending in a longitudinal direction thereof to have a C-shape in section, an outer diameter larger than an inner diameter of the wound part, and an engaging part protruding from an end thereof, said cylindrical spring member being disposed in the wound part in a contracted state and said engaging part being adapted to be attached to the other of the first member and the second member.

2. A hinge device according to claim 1, further comprising a bracket part protruding from one of the first member and the second member to which said coil spring with the cylindrical spring member therein is accommodated, and a shaft part for rotationally connecting the first and second members.

3. A hinge device according to claim 2, wherein said bracket part includes a cavity for retaining the coil spring and the cylindrical spring member therein, and an engaging portion for engaging the coupling part.

4. A hinge device according to claim 3, wherein said shaft part is integrally formed with the bracket part to protrude from an, outer end surface thereof and is rotatably attached to the other of the first member and the second member.

5. A hinge device according to claim 2, wherein said bracket part includes said shaft part protruding from one side to be rotatably attached to the other of the first member and the second member, a supporting shaft protruding from the other side thereof for supporting the coil spring and the cylindrical spring member thereon, and an engaging hole for receiving the coupling part so that the coupling part is immovably fixed to the bracket part.

6. A hinge device according to claim 3, wherein said bracket part further includes a shaft hole formed in one end thereof, said the shaft part passing through the shaft hole to rotationally connect the first member and the second member and supporting the coil spring and the cylindrical spring member thereof.

7. A hinge device according to claim 6, wherein said bracket part further includes an engaging hole for receiving the coupling part so that the coupling part is immovably fixed to the bracket part.
